(19) United States
(12) Patent Application Publication
(10) Pub. No.: US 2003/0235619 A1
(43) Pub. Date: Dec. 25, 2003

(54) Polymer-lipid delivery vehicles

(76) Inventors: Christine Allen, Toronto (CA); Murray Webb, North Vancouver (CA); Paul Tardi, Surrey (CA); Yumin Yuan, Toronto (CA)

Correspondence Address: Kate H. Murashige, Morrison & Foerster LLP, Suite 500, 3811 Valley Centre Drive, San Diego, CA, 92130-2332, US

(21) Appl. No.: 10/329,053

(22) Filed: Dec. 23, 2002

Related U.S. Application Data
(60) Provisional application No. 60/357,639, filed on Feb. 20, 2002.
(60) Provisional application No. 60/401,984, filed on Aug. 7, 2002.

(30) Foreign Application Priority Data
Dec. 21, 2001 (CA) CA 2,365,806

(51) Int. Cl. A61K 9/16; A61K 9/50

(52) U.S. Cl. 424/490

(57) ABSTRACT

Delivery vehicles comprising nanoparticles which are composed of: (a) a biodegradable hydrophobic polymer forming a core, and; (b) an outer amphiphilic layer surrounding the polymer core containing a stabilizing lipid are suitable for delivering active agents.

### TECHNICAL FIELD

This invention is directed towards vehicles comprising nanoparticles for delivery of drugs, especially hydrophobic drugs.

### BACKGROUND ART

Drug delivery vehicles including lipid-based delivery vehicle systems have been extensively developed and analyzed for their ability to improve the therapeutic index of drugs by altering the pharmacokinetic and tissue distribution properties of drugs. This approach is aimed at reducing exposure of healthy tissues to therapeutic agents while increasing drug delivery to a target site.

Considerable effort has been devoted to the development of novel approaches for the delivery and administration of organic active agents including hydrophobic drugs. The clinical utility and economic potential of hydrophobic drugs are well established. Highly water-insoluble organic drugs have significant solubility issues and generally require co-solubilization agents and/or premedication to ameliorate side effects encountered during administration and long infusion periods. Hydrophobic drugs, especially anti-neoplastic drugs, would thus benefit from increases in therapeutic activity arising from increased circulation lifetimes conferred by an appropriate delivery vehicle composition. Taxol® and etoposide are examples of hydrophobic drugs that would benefit from improved methods of delivery.

Delivery vehicle formulations of organic active agents often suffer the disadvantages that they exhibit “unsatisfactory entrapment efficiency”; poor stability; unacceptable delivery primarily to the liver and spleen; poor plasma pharmacokinetics; rapid dissociation of active agent; and anti-tumor activity that is modest or not improved at all (Sharma, et al., Cancer Letters, (1996) 107:265-272; Sharma, et al., Int. J. Cancer (1997) 71:103-107; Crosasso, et al., J. Controlled Release (2000) 63:19-30; and Cabanes, et al., Int. J. Oncol. (1998) 12:1035-1040).

A number of injectable drug delivery systems have been investigated as carriers of organic active agents including liposomes, microcapsules and microparticles. A significant obstacle to the use of these injectable drug delivery materials is the rapid clearance of the materials from the bloodstream by the monophagocytic system (MPS). Further obstacles are issues of effective size, stability and drug retention.

Significant effort has also been expended to formulate organic drug preparations using mixed micellar and emulsion type formulations, including the use of PEG-modified phospholipids to stabilize oil in water emulsions, in an attempt to ameliorate these disadvantages (Alkan-Onyuksel, et al., Pharm Res. (1994) 11:206-212; Lundberg, J. Pharm. Pharmacol. (1997) 49:16-21; Wheeler, et al., Pharm. Sciences (1994) 83:1558-1564).

Other polymeric drug delivery compositions have been proposed. For example, PCT publication WO 92/01477 describes a system comprising a polymeric material having a drug covalently bonded thereto through a pH sensitive covalent bond, which will release the drug at low pH. U.S. Pat. No. 4,610,868 describes a matrix material having a particle size in the range of 500 nm-100 μm which is composed of a hydrophobic compound and an amphipathic compound. The resulting “lipid matrix carriers” encapsulate biologically active agents and effect release from the matrix. U.S. Pat. No. 5,869,103 describes particulate compositions in the size range of 10 nm-200 μm where the particles are formed by combining emulsions of an active agent with mixtures of a biodegradable polymer and a water-soluble polymer. A number of such biodegradable and water-soluble polymers, including copolymers are described.

PCT publication WO 95/26376 describes polymer microspheres in the size range of 10 nm -2 mm which comprise spherical core particles of a non-water-soluble polymer and a surface layer which consists essentially of a water-soluble polymer which polymer may be coupled to polyethylene glycol (PEG). The polyethylene glycol is said to anchor the water-soluble polymer to the core particle. Although the applicants recognize the possibility of using such compositions in pharmaceutical applications, no preparations which involve incorporation of bioactive agents are described.

U.S. Pat. No. 5,145,684 describes particulate preparations wherein a crystalline drug substance is itself coated with a surface modifier for administration to subjects. Similarly, U.S. Pat. No. 5,470,583 describes nanoparticles having nonionic surfactants as a surface modifier associated with a charged phospholipid. In this case as well, the biologically active substance, itself having a particle size of <400 nm, is used as the core of the particles.

U.S. Pat. No. 5,891,475 describes drug delivery vehicles which contain hydrophilic cores such as those prepared from polysaccharides. The particles are treated to contain an external layer of fatty acids grafted onto the core by covalent bonds.

U.S. Pat. No. 5,188,837 describes microparticles which are generally in the size range of 1-38 μm which contain a solid hydrophobic polymer as a core and a phospholipid, such as phosphatidyl choline or lecithin as an exterior coating. According to this disclosure, other phospholipids such as phosphatidyl inositol and phosphatidyl glycerol are unworkable in this system. U.S. Pat. No. 5,543,158 discloses 1 nm-1 μm particles with polymeric cores and a surface layer of PEG, which may be linked covalently to a biologically active agent contained therein.

There are a number of publications in the open literature that also describe particulate drug delivery systems. For example, Perkins, W. R., et al., Int. J. Pharmaceut. (2000) 200:27-39 describe “lipocores” which are formed from a core of a poorly water-soluble drug surrounded by a PEG-conjugated lipid.

Gref, R., et al., Coll and Surf B: Biointerfaces (2000) 18:301-313 describe the nature of protein absorption onto PEG-coated nanoparticles formed from various polymers and copolymers, including polycaprolactone. Although it is recognized that such particles might be useful in pharmaceutical applications, only the particles themselves were studied. Lemoine, D., et al., Biomaterials (1996) 17:2191-2197 reports studies of various nanoparticles composed of, among other polymers, polycaprolactone. Again, while recognizing these as useful in delivery systems, only the particles themselves were studied.

Lamprecht, A., et al., Int. J Pharmaceut. (2000) 196:177-182 reports the study of the effect of the use of microfluidizers on the particle size of nanoparticles obtained using various hydrophobic polymers and copolymers.

Kim, S-Y., et al., J. Cont. Rel. (2000) 65:345-358 describe copolymeric nanospheres of Pluronic® with polycaprolactone (PCL). Nanospheres of Pluronic®/PCL block copolymers having an average diameter of <200 nm were loaded with endomethicin and evaluated with regard to cytotoxicity, drug release, drug loading efficiency and physical characteristics. The particles are formed entirely of the block copolymer.

Despite the substantial number of preparations of nanoparticles designed for drug delivery, an ideal composition has not been achieved. In order to perform successfully as a drug delivery composition, it is desirable that the nanoparticles be stable in the presence of serum or plasma (i.e., do not aggregate, precipitate, or bind to plasma proteins); that the particles can successfully be loaded with a significant amount of drug; and that the release of the drug is timed so as to maximize its successful delivery to the target tissue.

### DISCLOSURE OF THE INVENTION

The delivery vehicles described herein provide favorable pharmacokinetics and effective delivery of biologically active agents that are relatively insoluble in water. The delivery vehicles are emulsions of nanoparticles with a hydrophobic polymeric core which may contain the biologically active agent surrounded by a protective layer which is amphiphilic and thus prevents aggregation or precipitation of the particles and inhibits association of the particles with protein in plasma or serum. The vehicles may be prepared by a method previously not contemplated for the preparation of nanoparticles.

Thus, in one aspect, the invention is directed to a composition that comprises nanoparticles having at least one biodegradable hydrophobic polymer forming a core, and an outer amphiphilic layer surrounding the polymer core. The amphiphilic layer includes at least one stabilizing lipid. The outer layer need not be continuous. Preferably the nanoparticles have an average diameter of 50-300 nm.

The nanoparticles may further comprise an active agent within the polymeric core.

In another aspect, the invention is directed to methods of making these compositions, comprising the steps of:

(1) dissolving the component(s) of the amphiphilic layer in a first solvent system;

(2) dissolving an active agent and a hydrophobic, biodegradable polymer in a second solvent system,

said first solvent system comprising at least one organic component and an aqueous component or only an aqueous component; and said second solvent system comprising at least one organic component;

(3) combining the resulting solutions of Steps (1) and (2) and dispersing the resulting mixture by mechanical mixing;

(4) removing said organic components; and

(5) exchanging the remaining aqueous components with buffered solution.

Generally, this method allows for an encapsulation efficiency of the active agent in the range from about 50 to about 99%.

### BRIEF DESCRIPTION OF THE DRAWINGS

FIG. 1 is a graph showing the in vitro release kinetics of Taxol® from nanoparticles consisting of a PCL and Taxol® core with a lipid composition of DPPC/DSPC/DSPE-PEG2000 (45:45:10 mole ratio) in HBS.

FIG. 2 is a graph showing the in vitro release kinetics of Taxol® from nanoparticles consisting of a PCL and Taxol® core with a lipid composition of DPPC/DSPC/DSPE-PEG2000 (45:45:10 mole ratio) in serum.

FIG. 3 is a histogram showing the in vivo concentration of Taxol® one hour following intravenous administration of nanoparticles consisting of PCL and Taxol® core with a lipid composition of DPPC/DSPC/DSPE-PEG2000 (45:45:10 mole ratios) and DSPC/DSPE-PEG2000 (90:10 mole ratio) into female Balb/c mice. Results were compared against Taxol® formulated in Cremophor EL.

FIG. 4 is a graph showing the size of nanoparticles stabilized with DPPC/DSPC/DSPE-PEG2000 (45:45:10 mole ratio) and PVA and consisting of a PCL and Taxol® core as a function of the fraction of lipid (lipid includes DPPC/DSPC/DSPE-PEG2000) in the lipid and PVA mixture.

FIG. 5 is a histogram showing the in vivo concentration of Taxol® one hour following intravenous administration of nanoparticles, consisting of PCL and Taxol® core stabilized with DSPE-PEG550 and PCL-b-PEO, to Balb/c mice in comparison to Taxol® formulated in Cremophor EL. The drug to core PCL weight ratio was either 1:16 or 1:20.

### MODES OF CARRYING OUT THE INVENTION

The invention provides compositions which are useful in delivering biologically active agents wherein a hydrophobic core in which many biologically active agents are soluble is surrounded by a protective layer of at least one amphiphilic component. The amphiphilic component may be a stabilizing lipid or include a stabilizing lipid and may comprise an amphiphilic polymer, or several such polymers.

“Stabilizing lipids” include, but are not limited to, lipids that contain surface stabilizing polymers conjugated to the lipid headgroup. Preferably, the polymer conjugated to the lipid headgroup is hydrophilic. A preferred hydrophilic polymer-conjugated lipid is a polyethyleneglycol-conjugated lipid. The polymer making up the polymer-lipid conjugate can be a polymer that contains a backbone that allows it to associate with the core of the particle thereby enhancing the stability of the delivery vehicle (e.g., poly(vinyl alcohol) conjugated to a lipid).

Stabilizing lipids include some lipids that are not conjugated to a stabilizing polymer. Such lipids contain a negatively charged phosphate group shielded by a hydrophilic neutral moiety such as phosphatidylglycerol (PG) and phosphatidylinositol (PI).

The polymer core may comprise one or more lipophilic polymers or copolymers that are biodegradable. Suitable hydrophobic core polymers include those employed as the hydrophobic block of an amphiphilic copolymer. Suitable polymers making up the core include polycaprolactone (PCL), poly(d,1-lactide) (P(d,1-LA) or PLA), poly(β-Benzyl-l-aspartate), poly(Benzyl-l-glutamate) and polymers of a similar degree of hydrophobicity. The preferred molecular weight of the polymer is dependent on the nature of the polymer.

In preferred embodiments of the invention, the above described delivery vehicle incorporates one or more active agents. Preferably said agent is a water-insoluble drug such as Taxol®, an etoposide-compound, a camptothecin-compound and valrubicin or combinations thereof. Any biologically active agent may be included in the nanoparticles.

The preferred weight ratio of active agent to core polymer is from about 1:1 to about 1:50 of agent to polymer; as will be apparent from the description below, the ratio of active agent to core polymer is inherently limited by the nature of the composition. A preferred weight ratio of components for both the composition and method may include about a 1:30 active agent/polymer.

Preferably the components of the outer stabilizing layer, which layer may be discontinuous including stabilizing lipid(s), and the polymer within the polymer core are in a ratio sufficient to maintain the nanoparticles of an injectable size, from about 50 nm to about 300 nm, preferably less than 200 nm and to provide particles that are stable at temperatures in a range of from about 0° C. to about 45° C. Also, the components of the outer stabilizing layer and the polymer or polymers making up the polymer core are selected to optimize the entrapment efficiency and/or release profile of the active agent.

The delivery vehicles of the present invention may be used not only in parenteral administration but also in topical, nasal, subcutaneous, intraperitoneal, intramuscular, or oral delivery or by the application of the delivery vehicle onto or into a natural or synthetic inplantable device at or near the target site for therapeutic purposes or medical imaging and the like. Preferably, the delivery vehicles of the present invention are used in parenteral administration, most preferably, intravenous administration.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

Abbreviations

PEG: polyethylene glycol; PEG preceded or followed by a number: the number is the molecular weight of PEG; PEG-lipid: polyethylene glycol-lipid conjugate; DSPE-PEG2000: distearoylphosphatidylethanolamine derativized with polyethylene glycol with a molecular weight of 2000; PE: phosphatidylethanolamine; PC: phosphatidylcholine;

PI: phosphatidylinositol; PS: phosphatidylserine; DSPE: distearoylphosphatidylethanolamine; DSPC: distearoylphosphatidylcholine; DPPC: dipalmitoylphosphatidylcholine; DMPC: dimyristoylphosphatidylcholine; DMPE-PEG2000: dimyristoylphosphatidylethanolamine derivatived with polyethylene glycol with a molecular weight of 2000 daltons; EA: ethyl acetate; BA: benzyl alcohol; LA: d,1-lactide; P(d,1-LA) or PLA: poly(d,1-lactide); PVA: polyvinylalcohol; P1LA: poly(1-lactide); PBLA: poly(benzyl-1-aspartate); PCL: polycaprolactone; HBS: 20 mM HEPES, 150 mM NaCl; Tm: phase transition (or melting) temperature; Tg: glass transition temperature; AUC: area under the curve; RT: room temperature; v/v: volume-to-volume ratio; QELS: quasi-elastic light scattering.

Stabilizing Lipids

The term “stabilizing lipid” refers to lipids that enhance the stability of the nanoparticles by adhering to the surface of the particle through either covalent or non-covalent interaction with the core of the particle. The stabilizing lipid may or may not act as an emulsifier. In some embodiments, the stabilizing lipid, e.g., a polymer-lipid conjugate is non-covalently attached to a polymer within the polymer core.

Stabilizing lipids include lipids derivatized with a polymer, i.e., a lipid covalently joined at its polar head moiety to a polymer. The conjugated lipid may be any lipid described in the art for use in such conjugates such as phosphoglycerides, sphingolipids and ceramides. Preferably, the polymer is hydrophilic. The conjugate may be prepared to include a releasable lipid-polymer linkage such as a peptide, ester, or disulfide linkage. The conjugate may also include a targeting ligand.

The preferred hydrophilic polymer for conjugation is a biocompatible polymer characterized by a solubility in water that permits the polymer chains effectively to extend away from the nanoparticle surface out into the aqueous medium surrounding it. Examples of the hydrophilic polymer are polyalkylethers, such as polyethylene glycol (PEG), polymethylethylene glycol, polypropylene glycol, and polyhydroxypropylene glycol. Additional suitable polymers include polyvinylpyrrolidone, polyvinyl alcohol and polyacrylic acid. Preferably, the hydrophilic polymer has a molecular weight between about 350 and 5,000 Daltons.

The polymer making up the polymer-lipid conjugate also can be a polymer that contains a backbone that allows it to associate with the core of the particle thereby enhancing the stability of the delivery vehicle. An example of such a polymer is poly(vinyl alcohol).

Stabilizing lipids may also be lipids that are not conjugated to a stabilizing polymer. Such lipids include phosphoglycerides; glycolipids; sphingolipids such as sphingosine, ceramides, sphingomyelin, gangliosides and cerebrosides, and in particular phosphatidyl glycerol (PG) and phosphatidyl inositol (PI).

The phase transition temperature (Tm) or acyl chain length of the lipid contained in the stabilizing lipid has a significant effect on the stability of the delivery vehicle, especially at elevated temperatures. High Tₘ lipids enhance the stability of the lipid composition in comparison to low Tₘ lipids. At 37° C., high Tₘ lipids are in the gel phase while low Tm lipids are in the fluid phase.

Additional Stabilizing Components

Other lipids and components can also be included in the preparation of the delivery vehicle, such as cholesterol and cholesterol derivatives, e.g., ethoxylated cholesterol, cholesteryl hemisuccinate, cholesterol esters, and cholesterol hemisuccinate. Other components may include ergosterol, alpha-tocopherol, vitamin A, vitamin E; and phytosterols such as campesterol, beta-sitosterol, stigmasterol and derivatives thereof. They may also include various phosphatidyl choline molecules.

In addition to comprising a stabilizing lipid, the stabilizing layer surrounding the nanoparticles may further comprise one or more amphiphilic polymers (or copolymers) having a hydrophobic portion that can associate with the core of the particle (if the particle is prepared with a hydrophobic core). Example 5 describes the preparation of poly(caprolactone) (PCL) particles containing a surface layer of PCL-b-poly(ethylene oxide) and PEG-lipid.

Preferably, amphiphilic copolymers have hydrophobic portions of PCL, poly(d,1-lactide) (P(d,1-LA) or PLA), poly(β-Benzyl-l-aspartate), poly(Benzyl-l-glutamate) and polymers of a similar degree of hydrophobicity. The preferred molecular weight of the polymer is dependent on the nature of the polymer. The hydrophilic portion of the amphiphilic copolymer may include blocks such as polyethylene glycol (PEG), polymethylethylene glycol, polyhydroxypropylene glycol, polypropylene glycol, polyvinylpyrrolidone, polyvinyl alcohol, poly(vinylpyrrolidone) and polyacrylic acid.

Stabilizing polymers, such as poly(vinyl alcohol) PVA, that contain a hydrophobic backbone that can interact with the core (if the core is hydrophobic) can also be employed in addition to the stabilizing lipid (see Examples 3 and 4 for the preparation of PCL nanoparticles containing PVA/DSPE-PEG and PVA/DSPE-PEG/DPPC/DSPC).

Polymer Core

The term “polymer core” refers to the core of the nanoparticles and comprises one or more polymers, and is lipophilic and biodegradable. The polymer core may be semi-crystalline, crystalline or amorphous.

Examples of suitable polymers are polyesters such as polylactide (PLA or P(d,1-LA)), polyglycolide, polyhydroxybutyrate, polycaprolactone (PCL). Such polymers also include poly amino acids, polyanhydrides, polyorthoesters, polyphosphazines, poly(alpha.-hydroxy acids), polyphosphate esters, polyethylene terephalate, polyalkylcyanoacrylate and copolymers prepared from the monomers of these polymers.

Preferred polymers may include PCL and P(d,1-LA) homopolymers, di, tri and multiblock copolymers having a preferred molecular weight from about 5,000 to about 200,000 daltons. Where the choice of polymer is PCL, the molecular weight is preferably about 10,000 daltons. Where the choice of polymer is P(d,1-LA), the molecular weight is preferably about 100,000 daltons. Such polymers are well known biocompatible polymers that have been studied extensively for applications in drug delivery. The physical properties of P(d,1-LA) and PCL are quite different. The polymer P(d,1-LA) is known as a moderately hydrophobic amorphous polymer with a glass transition temperature (T_{g}) of approximately 50° C. while PCL is a hydrophobic semi-crystalline polymer with a T_{g} of −60° C. and a melting temperature (Tₘ) of 56° C. Both the T_{g} and Tₘ of the polymers are dependent on the molecular weight of the polymer. The Tₘ and T_{g} of PCL of were confirmed by differential scanning calorimetry (DSC) (Perkin Elmer). Optional polymers with an increased degree of hydrophobicity such as poly(B-benzyl-1-aspartate) (PBLA) may also be used.

Ratio of Stabilizing Lipid and Polymer Core in Combination

The stabilizing lipid layer and polymer making up the polymer core are used at a ratio sufficient to maintain the nanoparticles of an injectable size of from 300 nm to about 50 nm and that is stable at temperatures from 0° C. to about 45° C. Components at optimal ratios improve the entrapment efficiency and release profile of the encapsulated active agent. The components, in combination, have properties including, but not limited to, better stability at size diameters of from about 300 nm to about 50 nm at physiological conditions and temperatures, improved drug retention parameters and a non-bimodal distribution as indicated by Chi-squared values, (See Table 1).

Active Agent

The term “active agent” or “agent” as used herein refers to chemical moieties used in therapy or diagnosis and for which drug delivery in accordance with this invention is desirable. Included in this definition are therapeutic agents and imaging agents. Preferably, the active agent is “poorly soluble” in water or buffer. Delivery vehicles of this invention are particularly suitable for the delivery of poorly soluble active agents. The term “poorly soluble” with reference to an active agent in water or buffer means that the active agent has a solubility in the water or buffer of less than about 10 mg/mL. Any active agent may be used in the invention compositions.

A preferred therapeutic agent suitable for use in the present invention is an “anti-neoplastic agent.” The term “anti-neoplastic agent” refers to chemical moieties having an effect on the growth, proliferation, invasiveness or survival of neoplastic cells or tumours. Anti-neoplastic therapeutic agents include etoposide-compounds, antimicrotubule agents, camptothecin compounds, disulfide compounds, alkylating agents, antimetabolities, cytotoxic antibiotics and various plant alkaloids and their derivatives.

The term “etoposide-compound” also refers to both etoposide and derivatives of etoposide with a similar core structure including teniposide. Etoposide and teniposide are poorly water soluble (less than 10 mg/mL), are currently used in therapy for a variety of cancers, including testicular neoplasms, lung cancers, lymphomas, neuroblastoma, AIDS related Kaposi's Sarcoma, Wilms' Tumor, various types of leukemia, and others. Teniposide has also exhibited activity against bladder cancer, lymphomas, neuroblastoma, small cell lung cancer, and certain CNS tumors. Teniposide has not been studied as extensively as etoposide, but is presumed to have similar properties.

The term “antimicrotubule agent,” refers to agents that disrupt the normal function of the cellular microtubules. Included in this definition are the taxanes (paclitaxel and docetaxel are the representative agents of this class) and vinca alkaloids (vincristine, vinblastine and vinorelbine are other members of this class).

The term “camptothecin-compound” refers to camptothecin and derivatized forms of this plant alkaloid having topoisomerase inhibition activity, including topotecan, ironotecan, lurtotecan, 9-aminocamptothecin, 9-nitrocamptothecin and 10-hydroxycamptothecin, including salts thereof. Preferably the camptothecin-compound is camptothecin.

Methods of Preparation

The delivery vehicles of the invention comprising nanoparticles may be prepared using a number of conventional techniques known in the art. However, it is preferred that an improved preparation method be employed as will further be described below.

Various techniques are known for preparing aqueous colloidal dispersions containing small particles (nanoparticles), including salting out, emulsification-diffusion, nano-precipitation and emulsification-evaporation, which have in common that they involve the use of an organic solution, containing the small particle components. One conventional method of micro-encapsulating an agent to form a microencapsulated product is disclosed in U.S. Pat. No. 5,407,609. This method involves dissolving or otherwise dispersing agents, liquids or solids, in a solvent containing dissolved wall-forming materials, dispersing the agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres.

The most common method used for preparing polymer delivery vehicle formulations is the solvent evaporation method. This method involves dissolving the polymer and drug in an organic solvent that is completely immiscible with water (for example, dichloromethane). The organic mixture is added to water containing a stabilizer, most often poly(vinyl alcohol) (PVA). Using this solvent evaporation method with PVA requires a high energy shear force, (for example, sonication), that in itself has titanium contamination problems. Similar methods using organic solvent mixtures with water have also been problematic as these methods use completely immiscible solvents. These methods require excessive processing steps and high energy shear forces to produce particles of an acceptable sizes for intravenous administration.

Although the methods available in the art may be used, it is preferred to prepare the delivery vehicle compositions of the present invention in an improved process, further described as follows:

The initial step (Step (1)) is to dissolve a stabilizing lipid and optionally one or more components making up the surface stabilizing layer in a first solvent system comprising an aqueous component and one or more components that are miscible, partially miscible or immiscible with water and that are sufficient to solubilize the stabilizing lipid and additional components. Water miscible solvents that may be used in the first solvent system include acetone and alcohols, including short-chain alcohols, i.e., water miscible alcohols of less than four carbon atoms in length, such as ethanol, methanol, isopropyl alcohol. Partially water miscible solvents include ethyl acetate, isopropyl alcohol, benzyl alcohol, 2-butanone, 1-butanol, tetrahydrofuran, isopropyl alcohol, isopropyl acetate, propylene carbonate, methyl acetate. Water immiscible solvents that may be used include chloroform and dichloromethane. Combinations of water miscible, partially water miscible and water immiscible solvents may also be used.

In a preferred embodiment, the first solvent system is an aqueous component or a mixture of an aqueous component and a water miscible solvent, such as an alcohol, preferably short chain alcohol in a ratio of about 1:25 v/v (alcohol:water). Short chain alcohols are preferably selected from the group consisting of methanol, ethanol and isopropyl alcohol or combinations thereof. When a water miscible solvent is used, Step (1) is carried out at temperatures above the transition temperature of the stabilizing lipid having the highest phase transition temperature, preferably 5° C. to about 20° C. above the phase transition temperature of said lipid. Most preferably, Step (1) is carried out at 10° C. above the phase transition temperature of the lipid having the highest phase transition temperature.

Step (2) of the method is to dissolve the core polymer and active agent in a second solvent system containing one or more organic solvents that are miscible, partially miscible or immiscible with water and that are able to solubilize the active agent and core polymer. Water miscible solvents that may be used in the second solvent system include acetone and alcohols including short-chain alcohols; partially water miscible solvents include ethyl acetate, isopropyl alcohol, benzyl alcohol, 2-butanone, 1-butanol, tetrahydrofuran, isopropyl alcohol, isopropyl acetate, propylene carbonate and methyl acetate; water immiscible solvents include chloroform and dichloromethane. Combinations of water miscible, partially water miscible and water immiscible solvents may also be used in the second solvent system. The second solvent system preferably includes water immiscible and partially water miscible solvents. Preferably, the solvent system is selected from ethyl acetate, chloroform, propylene carbonate, benzyl alcohol, methyl acetate, 2-butanone, tetrahydrofuran, 1-butanol, isopropyl acetate or combinations thereof. Ethyl acetate and benzyl alcohol may be used in combination at a preferred ratio of about 1:1 v/v. The preferred choice of solvent or mixtures thereof making up the second solvent system can be chosen in accordance with the choice of active agent used.

Method related parameters that are adjusted to optimize particle stability, polydispersity, and drug leakage include the phase transition temperature of the lipids included in Step (1); ratios of solvents making up the second solvent system of Step (2); the ratio of the resulting solutions of Step (1) to Step (2) after admixing; and the rate and duration of subsequent mechanical mixing in Step (3). Additionally, components of the present method may be formulated according to specific requirements selected from the following (i) type of active agent used; (ii) the method of delivery of agent to a subject; (iii) the required release profile; and (iv) the purpose of use or target site chosen.

Resulting solutions of Step (1) and Step (2) may be at a ratio of about 10:1 to 1:30 v/v respectively after combining. Preferably, the ratio of the resulting solutions of Steps (1) and (2) are combined at a ratio 1:1 to 1:10 v/v respectively. A preferred ratio of combining the resulting solutions is 1:2 v/v [solution of Step (1) to solution of Step (2)] when ethyl acetate/benzyl alcohol mixtures and ethanol water mixtures are used.

Admixture of the resulting solutions of the two initial steps is followed by Step (3) which is mechanical mixing. A preferred method of mechanical mixing is homogenization over sonication and milling. The mixture is homogenized for a period of time that is dependent on the type or manufacture of homogenizer used.

Following mechanical mixing, the resulting solution may be diluted by the addition of aqueous solution, preferably water. Preferably, the mixture is diluted by slow addition of water while mixing. A preferred means of mixing is vortexing.

Following mechanical mixing, removal of the organic components of the first and second solvent system (Step (4)) is achieved by conventional techniques known to one of skill in the art for removal of such solvents. Preferred methods include rotary evaporation and stirring the solution at room or elevated temperatures and dialyzing. Preferably, removal of the organic components is by dialysis against water.

Subsequent to removal of organic components of the first and second solvent system, the remaining aqueous components are replaced with buffer solution. This step may be carried out by conventional techniques such as dialyzing, tangential flow, column chromatography and lyophilization. A preferred technique is by tangential flow. The pH of the buffer is preferably at physiological pH. This step may be carried out in order to concentrate the sample to a desired concentration.

The method may optionally further comprise a step of diluting the resulting solution after mechanical mixing. Dilution may be carried out by conventional techniques known to one of skill in the art. Preferably, dilution is carried out by slow addition of aqueous solution while mixing. A preferred method of mixing is vortexing. Preferably, the resulting solution of Step (3) is diluted with water.

Following removal of the first and second solvent systems, preferably, water is exchanged for a buffer solution. Buffer exchange may be carried out by conventional techniques such as dialyzing, tangential flow and lyophilization. The pH of the buffer is preferably at about physiological pH (for example; HBS, pH=7.4). Preferably, the solution is concentrated using a tangential flow apparatus (for example, MidGee™ Cross Flow Filter, AG Technology Corporation).

Several method related parameters may affect the stability, size, polydispersity, drug release profile and drug encapsulation, drug retention of the nanoparticle prepared. The parameters identified include: volume ratio of aqueous to organic phase, drug to polymer ratio, stabilizing lipid to polymer ratio, temperature of mixing and rate and duration of homogenization.

The size of the nanoparticles depends upon the ratio of stabilizing lipid and polymer making up the polymer core is also influenced by the encapsulated active agent. Particles of between 50 nm to 300 nm in diameter, are suitable for parenteral administration (U.S. Pat. No. 5,527,528). Sizing and polydispersity of particles may be determined by quasi-elastic light scattering (QELS, Nicomp 370 submicron particle sizer and) as exemplified below. Polydispersity may be indicated with Chi-square value χ² as indicated in Table 1. In a preferred embodiment, the nanoparticles of the present invention are prepared to have substantially homogenous sizes in a selected range.

Administering Delivery Vehicles

The drug delivery vehicle compositions of the present invention may be administered to warm-blooded animals, including humans. For treatment of human ailments, a qualified physician will determine how the compositions of the present invention should be utilized with respect to dose, schedule and route of administration using established protocols. The present invention allows higher dose loading with smaller dose volume, longer site-specific dose retention, a more rapid absorption of active drug substance, increased bioavailability of the drug, higher safety, efficacy and better patient compliance.

Delivery vehicles of the present invention are administered using methods that are known to those skilled in the art, including but not limited to compositions formulated for parenteral or subcutaneous injection, oral administration in solid, liquid or gel form, rectal or topical administration, and the like.

The compositions of the present invention may be administered parenterally. Typically, this will comprise a solution of the drug delivery vehicle suspended in a pharmaceutically acceptable solution, preferably an aqueous solution. A variety of aqueous vehicle carriers may be used, for example, (water, buffered water, 0.9% isotonic saline, 5% dextrose and the like). The term “parenteral” as used herein means intravenous, intra-arterial, intra-muscular, intra-peritoneal and to the extent feasible, intra-abdominal and subcutaneous.

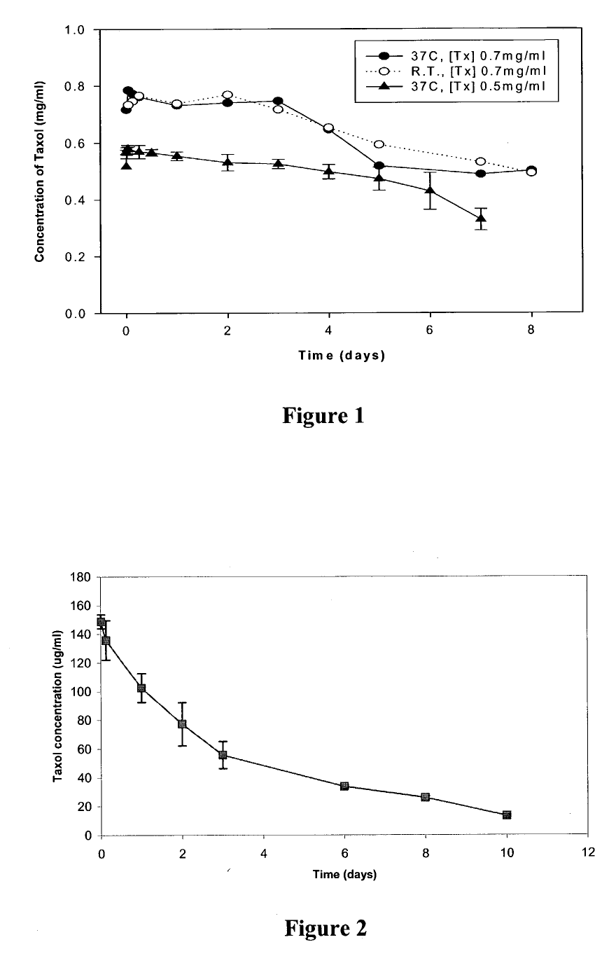

IN the examples below, Tables 1 and 2 and FIGS. 1 and 2 demonstrate that stability, drug release profile and the polydispersity at 37° C. can be held constant for up to a period of time in hours, days or weeks during release of drug. By altering the formulation of: the lipid composition; drug to polymer ratios; lipid to polymer ratios; or the second solvent system for the lipid and core polymer composition, each formulation is adapted to the specific requirements of required treatment with the most effective drug or combination of drugs thereof.

The following examples are offered to illustrate but not to limit the invention.

### EXAMPLES

Source of Materials

The homopolymers, poly(d,1-lactide) (P(d,1-LA) or PLA), poly(vinyl alcohol) (PVA) and poly(caprolactone) (PCL), and solvents were purchased from Sigma and PCL-b-PEO was purchased from Polymer Sources Inc. (Dorval,Quebec). Lipids were purchased from Northern Lipids and Avanti Polar Lipids.

Methods

Measurement of Size and Polydispersite of Nanoparticles

The size and polydispersity of the delivery vehicles were determined by quasi-elastic light scattering (QELs, Nicomp 370 submicron particle sizer operating at a wavelength of 632.8 nm).

Measurement of Drug Loading Capacity

The drug loading capacity was measured using a UV assay or by scintillation counting when radiolabeled Taxol® (¹⁴C) was used. The UV assay involved adding a 20 μL aliquot of the delivery vehicle solution to an acetonitrile/water mixture (8:2 ratio ACN:water) in order to precipitate the stabilizing lipid and polymer. The mixture was placed in the fridge for one hour and then centrifuged at 1000 g for 10 minutes. The amount of encapsulated Taxol® was calculated from the absorbance of the supernatant (230 nm) measured in a UV spectrophotometer. Standards ranging in concentration from 0.001-0.5 mg/mL Taxol® were treated as described above. The linear range for the calibration curve was from 0.001 to 0.05 mg/mL. Controls included empty delivery vehicles as well as the acetonitrile:water mixture alone. PCL levels were assumed to remain constant due to the hydrophobicity of the polymer.

Measurement of the Stability of the Delivery Vehicles at Room Temperature and at 37° C.

The stability of the lipid coated delivery vehicles were assessed by monitoring the solution for precipitation of the delivery vehicles as well as changes in particle size (measured as described above) during incubation at both room temperature (RT) and at 37° C. Precipitation, was noted to occur when a noticeable change in the solution occurred where the appearance had gone from having an iridescent/white homogeneous color to being clear with visible aggregates accumulated at the bottom of the dialysis bag or vial.

Measurement of in vitro Drug Release in Buffer or Serum at at Room Temperature (RT) and at 37° C.

To analyze the release of drug at RT and at 37° C., a 1 mL aliquot of the Taxol® containing-lipid/polymer delivery vehicle solution was placed in a dialysis bag (Spectrapor, mol. wt. cut off 50,000) and suspended in 1L of buffer (20 mM HEPES, 150 mM NaCl, (HBS) pH 7.4). At specific time intervals, a 10 μL (for scintillation counting) or 50 μL (for UV assay) aliquot was removed from the dialysis bag to determine drug levels. When calculating Taxol-release, drug/core PCL levels were determined and it was assumed that the PCL core of the nanoparticles remained intact.

### Example 1

The Preparation of PCL Nanoparticles Stabilized by DPPC/DSPC/DSPE-PEG Mixtures

Nanoparticle systems containing DPPC/DSPC/DSPE-PEG2000 (45:45:10 mole ratio) and incorporating Taxol® were prepared using the method of the present invention. Poly(caprolactone) (PCL) was selected as the hydrophobic polymer making up the core of the particle. The nanoparticles were coated with a stabilizing lipid/PEG-lipid mixture during particle formation. In this method, the polymer and drug are dissolved in a solvent that is partially miscible with water and then mixed with an aqueous solution containing the lipid. The mixture is then homogenized, diluted with water while vortexing and dialyzed. In order to prepare lipid-coated nanoparticles by this method, it was necessary to dissolve the stabilizing lipid (lipid and PEG-lipid solution) in an ethanol/water mixture rather than water alone. The stability of the nanoparticles was assessed by measuring the size and polydispersity of the particles; as well, the in vitro release of Taxol® from the particles in buffer and serum was determined.

DPPC, DSPC and DSPE-PEG2000 lipids (20 mg) were dissolved in 2 mL of an ethanol/water mixture of 0.25:1 v/v at a mole ratio of 45:45:10 and the resulting solution was heated to 65° C. In a separate tube, poly(caprolactone) (PCL) (20 mg) and Taxol® were dissolved at a 1:30 weight ratio in 1 mL of a 1:1 ethyl acetate/benzyl alcohol (organic mixture) solution. The above two solutions were combined at a 1:2 v/v of polymer-Taxol mixture/lipid mixture. The resulting solution was then homogenized (using a Polytron™ homogenizer) for 3 minutes and further diluted by the slow addition of water while vortexing. The solution was stirred at room temperature and then dialyzed against water for 8 hours to remove the organic solvent. Following the dialysis procedure, the water was exchanged for 20 mM HEPES, 150 mM NaCl (HBS buffer), pH 7.4 and the solution was concentrated using a tangential flow apparatus (MidGee™ Cross Flow Filter, AG Technology Corporation). The stability of the resulting Taxol-containing nanoparticles during dialysis in buffer at room temperature (RT) and 37° C. was measured according to the above-described method by monitoring the solution for precipitation of the nanoparticles as well as changes in particle size. The mean nanoparticle diameter obtained by this method, as well as the polydispersity as measured above at 37° C., are indicated in Table 1 below. As evidenced by the results in Table 1, the size of the nanoparticles remained roughly constant throughout the time course measured.

TABLE 1

| Temp 37° C. | Size (nm) | Chi-square value X² |
|---|---|---|
| Before cone. | 107.6 | 0.19 |
| After conc. | 102.5 | 0.21 |
| Day 1 | 100.3 | 0.32 |
| Day 2 | 100.0 | 1.52 |
| Day 3 | 104.4 | 0.12 |
| Day 4 | 94.6 | 0.46 |
| Day 8 | 100.9 | 0.72 |
| Day 9 | 111.2 | 0.62 |
| Day 10 | 100.0 | 0.82 |

To analyse the release of drug in buffer at room temperature and at 37° C., a 1 mL aliquot of the Taxol-containing lipid/nanoparticle solution was placed in a dialysis bag (Spectrapor, mol. wt. cut off 50,000) and suspended in 1 L of HBS, pH 7.4 as specified in the Methods. At specific time intervals, a 10 μL (for scintillation counting) or 50 μL (for UV assay) aliquot was removed from the dialysis bag over a period of time. The initial Taxol concentration in the nanoparticle preparations was 0.5 or 0.7 mg/mL. The results summarized in FIG. 1 show that on day 7, the Taxol concentration was reduced to about 0.3 mg/mL from an initial concentration of 0.5 mg/mL when dialyzed at 37° C. against HBS buffer. Samples with an initial concentration of 0.7 mg/mL resulted in a Taxol concentration of about 0.6 mg/mL after being dialyzed for 8 days at room temperature and 37° C.

The in vitro release of Taxol in serum at 37° C. from the DPPC/DSPC/DSPE-PEG2000 coated PCL nanoparticles was also determined by the addition of 0.5 mL of the particles and 0.5 mL of serum to a dialysis bag and dialysing against HBS, pH 7.4. At the specified time points (see FIG. 2), 10 μL samples were removed and the Taxol concentrations were determined. Results depicted in FIG. 2 indicate that the release of Taxol from the nanoparticles in serum occurred gradually over the time course measured (10 days). After 10 days of incubation, roughly 10 pg of Taxol remained thus demonstrating that the nanoparticles can prolong the plasma drug residence time for up to ten days.

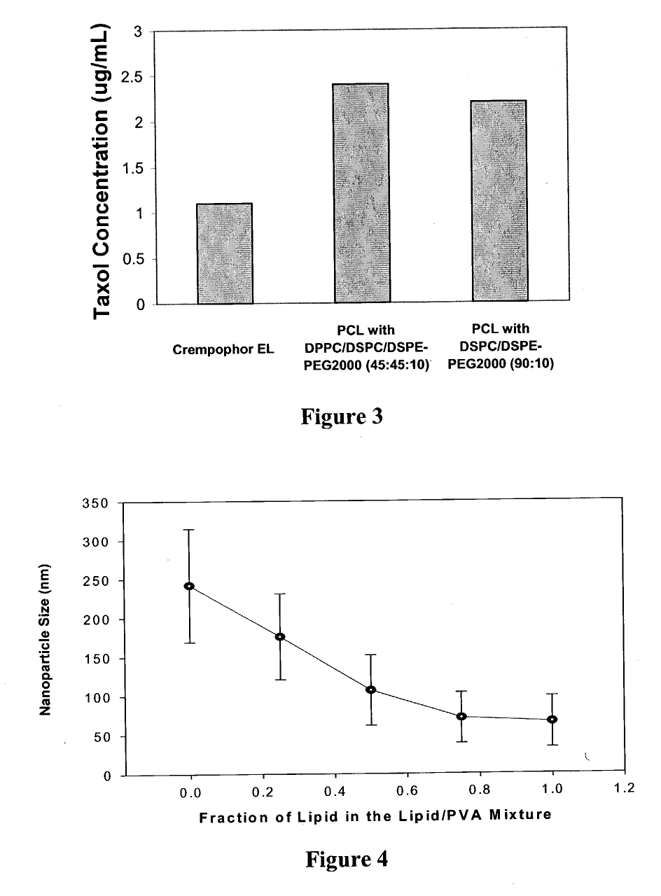

The ability of DSPC/DPPC/DSPE-PEG coated PCL particles and Cremophor EL to enhance the levels of Taxol in vivo in the blood compartment was compared. Cremophor EL is the current, gold-standard formulation used for the delivery of hydrophobic drugs such as Taxol and consists a mixture of glycerol-polyethylene glycol ricinoleate. Female Balb/c mice were administered 5.0 mg/kg Taxol, 133 mg/kg total lipid, and 133 mg/kg PCL for the nanoparticle preparations and 5.0 mg/kg Taxol in Cremophor EL. The data represents the mean Taxol concentration from 3 mice for each delivery system. As evidenced in FIG. 3, the PEG-lipid/lipid coated PCL nanoparticles containing DSPC/DPPC/DSPE-PEG2000 (45:45:10 mole ratio) and DSPC/DSPE-PEG2000 (90:10 mole ratio) as the surface stabilizer both displayed a greater than two fold increase in Taxol concentrations in the plasma at one hour post intravenous administration.

These results thus indicate that lipid-stabilized nanoparticles can be used as a superior alternative to Cremophor EL for the delivery of hydrophobic agents as demonstrated by the ability of the nanoparticles to increase the blood residence time of the drug. Furthermore, lipid-coated nanoparticles may not exhibit the undesirable patient toxicity that Cremophore EL formulations presently display.

### Example 2

PCL Nanoparticles cannot be Stabilized in the Absence of Stabilizing Lipid such as PEG-Lipid

In order to determine whether the presence of a stabilizing lipid such as DSPE-PEG was required for the preparation of lipid-coated PCL and PLA nanoparticles, nanoparticles were prepared in the absence of PEG-lipid, employing only DMPC or DPPC as the lipid coating.

The method was repeated in the same manner as in Example 1 to prepare PLA nanoparticles coated with DMPC and PCL particles coated with DPPC. PLA or PCL was dissolved with Taxol® at a 1:30 weight ratio in a 1 mL solution of 1:1 v/v benzyl alcohol:ethyl acetate. For the preparation of both nanoparticles, the drug and polymer precipitated out during the general procedure and therefore no size measurement could be obtained. These results thus indicate that stabilizing lipids, such as PEG-lipid conjugates are required in these systems to produce PCL or PLA nanoparticles that are stable in vitro. Examples of stabilizing lipids that, in addition to PEG-lipids, may potentially be suitable for inclusion in nanoparticles of this invention include those with a negatively charged phosphate group shielded by a hydrophilic neutral moiety, such as phosphatidylglycerol and phosphatidylinositol.

### Example 3

The Preparation of PCL Nanoparticles Stabilized by PVA/DSPE-PEG Mixtures

The inventors next examined whether the in vitro stability of the PEG-lipid-coated nanoparticles could also be enhanced by the addition of a hydrophilic polymer surface stabilizer, poly(vinyl alcohol) (PVA). PVA contains a hydrophobic, hydrocarbon backbone that allows it to stably associate with the hydrophobic core of the particles, while the hydrophilic portion of the polymer extends into the aqueous medium. PVA of a molecular weight of 10,000 g/mole was employed in order to minimize the viscosity of the system during particle preparation, as higher molecular weight PVA may result in an increase of the solution viscosity leading to an increase in particle size.

Stock solutions of 100 mg/mL PCL in ethyl acetate, 10 mg/mL Taxol® in ethyl acetate and 5% w/w poly(vinyl alcohol) (PVA) in water were prepared. A DSPE-PEG750 stock solution at a concentration of 100 mg/mL was prepared in ethanol. An organic solution was prepared by combining the PCL stock (200 μL) with 67 μL of the Taxol stock solution and 733 μL of ethyl acetate. In a separate vial, 300 μL of PVA and 50 μL of DSPE-PEG750 plus 50 μL of ethanol and 1.6 mL of water were mixed together to form an aqueous solution. The aqueous and organic solutions were combined and homogenized to create a nanoparticle dispersion. Following homogenization, a further 8 mL of water was added to the solution while vortexing. The solution was subsequently stirred at room temperature for 3-4 hours and then dialyzed against water overnight. Tangential flow was then used to concentrate the solution and exchange the water for buffer. The resulting nanoparticles contained a 1:30 drug to PCL weight ratio. The mean diameter of the particles was determined to be about 100 nm. PCL particles stabilized with PVA/PEG-lipid were also prepared according to the above procedure to have a 1:20 drug to PCL weight ratio.

The in vitro drug release and stability of PVA/DSPE-PEG containing PCL nanoparticles was determined in serum and the results are presented in Table 2 below. These results were compared with drug release and stability properties of PCL nanoparticles stabilized with DSPC/DPPC/DSPE-PEG2000 and lacking PVA (see Example 1). For in vitro release studies in serum, 500 μL of the nanoparticles were combined with 500 μL of serum and placed in a dialysis bag and dialyzed against 1 L of HBS buffer, pH 7.4. At specific time points, an aliquot of solution was removed from the dialysis bag and assayed for Taxol concentration by using both UV and HPLC. The stability of the nanoparticles was determined as set forth in the Methods.

TABLE 2

| | Drug load | | | Drug |
|---|---|---|---|---|
| | (Taxol/PCL | Drug | Stability | Retention |
| | weight | loading | at 37° C. in | after 24 |
| Formulation | ratio) | efficiency | serum | hours |
| PCL with DSPC/ | 1:30 | 92% | 10 days | 68% |
| DPPC/DSPE- | | | | |
| PEG2000 | | | | |
| PCL with PVA/ | 1:30 | 89% | 72 hours | 41% |
| DSPE-PEG750 | | | | |
| PCL with PVA/ | 1:20 | 93% | 24 hours | 0% |
| DSPE-PEG750 | | | | |

As indicated in Table 2, near complete drug incorporation was observed for all systems. As well, all particles displayed extended stability during incubation in serum at 37° C. as the particles, in all three cases, were stable for at least 24 hours. PCL particles stabilized by DSPC/DPPC/DSPE-PEG2000 displayed a drug retention of about 68% after a 24-hour incubation period in serum in vitro demonstrating that drug can be retained well over this time course. Particles prepared with PVA/DSPE-PEG750 at a Taxol/polymer weight ratio of 1:30 displayed about 41% drug retention at the 24-hour time point. PVA/PEG750-lipid particles containing a drug/polymer ratio as high as about 1:20 could be prepared; this is in contrast to previous results using a DSPE-PEG2000/DPPC/DSPC stabilizing mixture where it was only possible to prepare particles with drug/polymer ratio of 1:30. These particles containing PEG750 at a 1:20 ratio, however, displayed negligible Taxol retention at the 24-hour time point.

In addition to preparing PCL nanoparticles coated with PVA and DSPE-PEG750 as a stabilizer, particles were also prepared with DSPE-PEG550. Short chain PEG polymers, such as PEG550, can be employed to decrease the rate of exchange of the PEG-conjugate from the lipid layer. The procedures described above to prepare PCL particles stabilized with PVA/DSPE-PEG750 were repeated except the organic solution contained a mixture of 200 μL PCL stock solution, 100 μL Taxol stock and 700 μL of ethyl acetate.

### Example 4

The Preparation of PCL Nanoparticles Stabilized with PVA/DSPE-PEG/Lipid Mixtures

In addition to preparing particles containing a surface coating of PEG-lipid and PVA, PCL nanoparticles containing a mixture of PEG-lipid/PVA/DSPC/DPPC were also prepared. The effect of increasing levels of PEG-lipid/lipid to act as an emulsifier, by decreasing particle size, was also examined.

PCL stock of 100 mg/mL and a Taxol® stock of 10 mg/mL were prepared in ethyl acetate. A PVA stock of 5 % w/w was prepared in water and a solution of DSPC/DPPC/DSPE-PEG2000 at a 45:45:10 mole ratio was prepared in ethanol (100 mg/mL). An organic solution of drug and core polymer was prepared by combining 200 μL of the PCL solution, 100 μL of the Taxol® stock solution and 700 μL of the ethyl acetate solution. 0 to 400 μL of the aqueous PVA stock solution was combined with 200 to 0 μL of the lipid solution and the final volume was adjusted to 2 mL with water. The aqueous and organic solutions were combined and homogenized as set forth in Example 1. Subsequent to homogenization, 8 mL of water was added to the solution followed by dialysis and exchange into buffer as described in Example 1. These procedures resulted in nanoparticles of a 1:20 drug to PCL weight ratio.

As shown in FIG. 4, increases in the lipid (DSPC/DPPC/DSPE-PEG2000) to PVA ratio resulted in decreases in particle size. A 1:1 weight ratio of lipid to PVA enabled the preparation of nanoparticles that were 100 nm in size. In the absence of lipid/PEG-lipid (ie. PVA alone), the nanoparticles were 250 nm in diameter, thus the lipid mixture acts to reduce the size of the nanoparticles.

### Example 5

The Preparation of PCL Nanoparticles Stabilized by PCL-b-PEO/DSPE-PEG Mixtures

The inventors also utilized the amphiphilic block co-polymer, polycaprolactone-b-poly(ethylene oxide) (PCL-b-PEO), as a surface stabilizing agent with the goal of further increasing the stability of the PEG-lipid-coated PCL nanoparticles. The hydrophobic block of the copolymer is likely to interact strongly with the PCL core and thereby remain stably associated with the surface of the nanoparticle. The in vivo release kinetics of Taxol® from nanoparticles containing a mixture of PEG-lipid and PCL-b-PEO at two different ratios was compared to Taxol release from the gold-standard of its delivery, Cremophor EL.

Stock solutions of PCL in ethyl acetate (40 mg/mL), Taxol® in ethyl acetate (10 mg/mL) and PCL-b-PEO in ethyl acetate (50 mg/mL) were prepared. As well, a stock solution of DSPE-PEG550 was prepared at 50 mg/mL in ethanol. An organic solution containing 500 μL of the PCL stock, 300 μL of the PCL-b-PEO stock and 125 μL of the drug stock and 75 μL of ethyl acetate was prepared and combined with an aqueous solution containing 100 μL of DSPE-PEG550 stock plus 1.9 mL of water. The resulting mixture was homogenized, dialyzed and exchanged into buffer as described in Examples 1 and 2. These procedures resulted in nanoparticles of a mean diameter of about 100 nm with a 1:16 drug to PCL weight ratio. A similar procedure was used to generate nanoparticles with a 1:20 drug to PCL weight ratio.

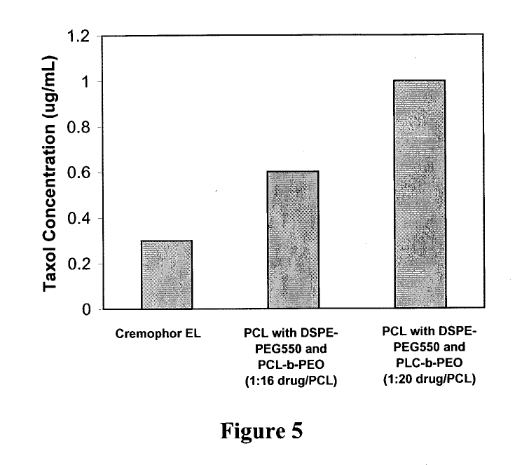

The ability of PCL-b-PEO/DSPE-PEG550 stabilized nanoparticles and Cremophor EL to increase the levels of Taxol in the blood compartment was compared at two different Taxol/core PCL weight ratios. Female Balb/c mice were administered 5.0 mg/kg Taxol®, 25 mg/kg total lipid, and 100 mg/kg core PCL for a drug to core PCL weight ratio of 1:16 and 5.0 mg/kg Taxol®, 20 mg/kg total lipid and 80 mg/kg core PCL for nanoparticles loaded at a 1:20 ratio (Taxol/core PCL). The data represents the mean Taxol concentration from 3 mice for each delivery system. As shown in FIG. 5, the PCL-b-PEO/DSPE-PEG550 coated PCL nanoparticles displayed a two fold increase in Taxol concentrations in the plasma at one hour post intravenous administration when loaded at a 1:16 Taxol/core PCL weight ratio and more than a three fold increase when loaded at a 1:20 ratio.

As well, the above-described procedures that were employed to prepare PCL nanoparticles stabilized with PCL-b-PEO/DSPE-PEG were slightly varied by preparing the Taxol® stock solution (10 mg/mL) in chloroform instead of ethyl acetate. These procedures also resulted in 100 nm nanoparticles of a 1:20 drug to core PCL weight ratio. Thus, this method of preparation is not limited to the use of solvents that are miscible with water (e.g. ethyl acetate, benzyl alcohol). Drugs of interest that are not soluble in chloroform may also be incorporated into these nanoparticles.

1. A delivery vehicle comprising nanoparticles which nanoparticles are composed of:
(a) a biodegradable hydrophobic polymer forming a core; and
(b) an outer continuous or discontinuous amphiphilic layer surrounding the polymer core, wherein said layer comprises one or more stabilizing lipids.

2. The delivery vehicle of claim 1 wherein said nanoparticles further comprise:
(c) a pharmaceutical that comprises at least one active agent.

3. The delivery vehicle of claim 1, wherein the polymer core comprises poly(caprolactone) (PCL).

4. The delivery vehicle of claim 3, wherein said PCL has a molecular weight from about 5,000 to about 45,000 daltons.

5. The delivery vehicle of claim 4, wherein PCL has a molecular weight of about 10,000 daltons.

6. The delivery vehicle of claim 1, wherein the polymer core comprises poly(d,1-lactide) (PLA).

7. The delivery vehicle of claim 6, wherein said PLA has a molecular weight from about 5,000 to about 200,000 daltons.

8. The delivery vehicle of claim 7, wherein said PLA has a molecular weight of about 100,000 daltons.

9. The delivery vehicle of claim 1, wherein the polymer core comprises di, tri or multi-block copolymers, or combinations thereof.

10. The delivery vehicle of claim 2, wherein said at least one active agent is an anti-neoplastic agent.

11. The delivery vehicle of claim 10, wherein said anti-neoplastic agent is selected from the group consisting of Taxol®, etoposide, camptothecin, valrubicin and podophylotoxins or functionally equivalent derivatives or combinations thereof.

12. The delivery vehicle of claim 2, wherein the polymer and pharmaceutical are in a ratio sufficient to maintain polymer association with said pharmaceutical.

13. The delivery vehicle of claim 12, wherein the pharmaceutical/polymer weight ratio is from about 1:1 to about 1:50.

14. The delivery vehicle of claim 1, wherein the stabilizing lipid is a polymer-conjugated lipid.

15. The delivery vehicle of claim 14 wherein the polymer-conjugated lipid is a PEG-lipid conjugate.

16. The delivery vehicle of claim 15, wherein the PEG-lipid conjugate is non-covalently attached to the polymer core.

17. The delivery vehicle of claim 1, wherein the stabilizing lipid is selected from the group consisting of phosphatidylglycerol and phosphatidylinositol.

18. The delivery vehicle of claim 1, wherein the average diameter of the nanoparticles is about 50-300 nm.

19. The delivery vehicle of claim 18, wherein said diameter is about 50-200 nm.

20. The delivery vehicle of claim 19, wherein said diameter is about 50-150 nm.

21. The delivery vehicle of claim 1, wherein the nanoparticles further comprise a phosphatidylcholine.

22. The delivery vehicle of claim 21, wherein the phosphatidylcholine comprises two fatty acids, each acyl chain being the same or different, at least one of said acyl chains having more than 6 carbon atoms.

23. The delivery vehicle of claim 22, wherein the fatty acids are stearoyl and/or palmitoyl.

24. The delivery vehicle of claim 1, wherein the amphiphilic layer further comprises an amphiphilic polymer.

25. The delivery vehicle of claim 24, wherein the amphiphilic polymer is poly(caprolactone)-PEG.

26. The delivery vehicle of claim 1, wherein the amphiphilic layer further comprises poly(vinyl alcohol).

27. A method of preparing a delivery vehicle comprising nanoparticles, which nanoparticles are composed of:
(a) a biodegradable hydrophobic polymer forming a core; and
(b) an outer amphiphilic layer surrounding the polymer core, wherein said layer comprises one or more stabilizing lipid; and
(c) a pharmaceutical that comprises at least one active agent;
which method comprises the steps of:
(1) dissolving the component(s) of the amphiphilic layer separately or together in a first solvent system;
(2) dissolving an active agent and a hydrophobic, biodegradable polymer separately or together in a second solvent system,
wherein the first solvent system comprises an aqueous component or one or more organic components and an aqueous component and the second solvent system comprises at least one organic component;
(3) combining the resulting solutions of Steps (1) and (2) and dispersing the resulting mixture by mechanical mixing;
(4) removing said organic components; and
(5) exchanging the remaining aqueous component with buffered solution.

28. The method of claim 27 further comprising the step of diluting the resultant of Step (3) with an aqueous solution.

29. The method of claim 27, wherein the polymer of Step (2) comprises a polyester polymer.

30. The method of claim 29, wherein the polyester polymer is poly(caprolactone).

31. The method of claim 30, wherein poly(caprolactone) has a molecular weight from about 5,000 to about 45,000 daltons.

32. The method of claim 29, wherein the polymer core comprises poly(d,1 lactide).

33. The method of claim 32, wherein poly(d,1 lactide) has a molecular weight of 5,000 to 200,000 daltons.

34. The method of claim 27, wherein the nanoparticles further comprise DPPC and/or DSPC.

35. The method of claim 27, wherein the stabilizing lipid comprises a lipid/PEG-lipid combination.

36. The method of claim 27, wherein Step (1) temperatures are elevated above the phase transition temperature of component (a) or (b).

37. The method of claim 27, wherein the second solvent system comprises a partially water miscible solvent or mixtures thereof.

38. The method of claim 37, wherein the solvent is selected from the group consisting of ethyl acetate, propylene carbonate and benzyl alcohol or combinations thereof.

39. The method of claim 27, wherein the first solvent system comprises a short-chain alcohol.

40. The method of claim 39, wherein the short-chain alcohol is ethanol.

41. The method of claim 27, wherein the nanoparticles are about 50-300 nm in diameter.

42. The method of claim 27, wherein the amphiphilic layer further comprises an amphiphilic polymer.

43. The method of claim 42, wherein the amphiphilic polymer is poly(caprolactone)-PEG.

44. The method of claim 27, wherein the amphiphilic layer further comprises poly(vinyl alcohol).
